(19) United States
(12) Patent Application Publication
(10) Pub. No.: US 2015/0010986 A1
(43) Pub. Date: Jan. 8, 2015

(54) Ettlia sp. Strain Having Superior Carbon Dioxide Fixation Ability and Lipid Producing Ability and Use Thereof

(71) Applicant: Korea Research Institute of Bioscience and Biotechnology, Daejeon (KR)

(72) Inventors: Hee Mock Oh, Daejeon (KR); Chan Yoo, Daejeon (KR); Gang Guk Choi, Daejeon (KR); Hee Sik Kim, Daejeon (KR); Hyun Joon La, Daejeon (KR); Chi Yong Ahn, Daejeon (KR)

(21) Appl. No.: 14/377,228

(22) PCT Filed: Apr. 4, 2012
(86) PCT No.: PCT/KR12/02546

§ 371 Date: Aug. 7, 2014

(30) Foreign Application Priority Data
Feb. 8, 2012 (KR) 10-2012-0012888

(51) Int. Cl. C12N 1/12

(52) U.S. Cl. CPC C12N 1/12
USPC 435/257.1

(57) ABSTRACT

Provided are a new microalgae strain and a use thereof, and more particularly, Ettlia sp. YC001 (KCTC 12109BP) having high carbon dioxide fixability, lipid productivity and carotenoid productivity, and a use thereof. The strain may be used for producing high quality biodiesel by controlling a lipid content and a composition ratio of a fatty acid according to culture conditions and/or culture time, and may be easily used for industrial uses, for examples, cosmetics, health foods, and medicines since large amounts of carotenoid and pigments are accumulated in cells.

### TECHNICAL FIELD

The present invention relates to novel microalgae, an Ettlia sp. strain and a use thereof, and more particularly, to Ettlia sp. YC001 (KCTC 12109BP) having high carbon dioxide fixability, lipid productivity and carotenoid productivity, and a use thereof

### BACKGROUND ART

As measured by the European Space Agency, over 300 million tons of excessive carbon dioxide is emitted into the air annually, and an amount of the carbon dioxide in the air is consistently increasing. Since the carbon dioxide in the air causes global warming, recently, studies on methods of reducing the carbon dioxide in the air through carbon capture and storage (CCS) have been actively progressing.

Meanwhile, due to depletion of coal and high oil prices, the importance of development of alternative energy that can replace fossil fuels such as petroleum or coal is increasing. Alternative energy currently in use includes hydroelectric power, nuclear power, wind power, tidal power, and solar powder. However, hydroelectric power causes environmental destruction by dam construction, nuclear power causes problems of treatment of radioactive wastes and stability, and wind power, tidal power and solar power using natural energy generate small amounts of energy, and have various problems including unstable supply of energy according to environmental conditions. Therefore, recently, a method of using algae having high carbon dioxide fixability, and capable of being used as biofuel has been receiving much attention.

Since approximately 14 million tons of algae are produced over the world annually and utilize the sea, a usable cultivation area is wide, and since an annual absorption rate of carbon dioxide is 5 to 7 times higher than lignocelluloses, an annual reduction rate of green house gas is also very high. In addition, since there is no lignin component that must be removed to be used as a biofuel, a process of manufacturing a biofuel is simple, and a total energy conversion ratio is high.

Algae are widely classified into macroalgae and microalgae. Here, the microalgae are living organisms having no distinguishable roots, stems or leaves that inhabit fresh water or seawater, have chlorophyll and perform photosynthesis, and contain vegetable fatty acids, proteins, minerals and all types of vitamins, and thus are known to be useful for humans. In addition, approximately 16 to 30% of all components in the microalgae is generally lipids or oil, and therefore biodiesel can be produced using a biomass thereof.

As described above, recently, there is a demand for development of microalgae strains having high carbon dioxide fixability and high lipid contents, which can be industrially utilized.

### DISCLOSURE

### Technical Problem

The present invention is provided to solve conventional technical problems, and therefore it is directed to providing novel microalgae strains having high carbon dioxide fixability and lipid productivity, and a use thereof.

However, technical objects accomplished by the present invention are not limited to the above-described objects, and thus other objects should be clearly understood from the following descriptions by those of ordinary skill in the art.

### Technical Solution

One aspect of the present invention provides an Ettlia sp. strain deposited under the Accession No. KCTC 12109BP.

In one embodiment of the present invention, the strain has an 18S rDNA sequence of SEQ. ID. NO: 3.

In another embodiment of the present invention, the strain has a lipid content of 30 to 67% of a dry weight.

In still another embodiment of the present invention, the strain has carotenoid productivity.

In yet another embodiment of the present invention, the strain has resistance from pH 6 to 11.

In yet another embodiment of the present invention, the strain is cultured under the condition of 15 vol % of carbon dioxide.

In yet another embodiment of the present invention, the strain is cultured for 3 to 60 days.

Another aspect of the present invention provides a composition for producing biodiesel, which includes the strain or a homogenate thereof.

Still another aspect of the present invention provides a composition for producing carotenoid, which includes the strain or a homogenate thereof.

Yet another aspect of the present invention provides a composition for foods, which includes the strain or a homogenate thereof.

Yet another aspect of the present invention provides a composition for cosmetics, which includes the strain or a homogenate thereof.

### Advantageous Effects

Novel microalgae according to the present invention, that is, an Ettlia sp. strain, has a high lipid content, is advantageous to grow in a wide pH range, and thus can be used industrially. In addition, the strain has very high photosynthesis efficiency, resulting in excellent carbon dioxide reducing efficiency and biomass productivity, and can be used to produce high quality biodiesel and other useful materials including an antioxidant material such as carotenoid by controlling culture conditions and/or a culture time. Accordingly, the strain is expected to be applied to various bio materials such as foods, cosmetics, etc. Since a morphological characteristic and a procedure of differentiation of cells make a clear distinction according to a concentration of carbon dioxide and a culture time, the strain is expected to be used in various physiological or genetic engineering studies of microalgae.

### DESCRIPTION OF DRAWINGS

FIG. 1 shows optical microscopic images of 12 types of microalgae separated from environment samples;

FIG. 2 shows optical microscopic images of Ettlia sp. YC001 of the present invention having excellent growth in a high concentration of carbon dioxide and a high lipid content;

FIG. 3 is a diagram showing an 18S rDNA sequence of Ettlia sp. YC001;

FIG. 4 is a growth curve of Ettlia sp. YC001 according to a concentration of carbon dioxide;

FIG. 5 is a diagram showing comparison of lipid contents on day 8 and day 16 of the culture of Ettlia sp. YC001 according to a concentration of carbon dioxide;

FIG. 6 is a diagram showing comparison of the growth rate, lipid content, and lipid productivity of Ettlia sp. YC001 according to a concentration of carbon dioxide;

FIG. 7 is a diagram showing comparison of lipid contents on day 8 and day 16 of the culture of Ettlia sp. YC001 according to a concentration of carbon dioxide;

FIG. 8 is optical microscopic images and a diagram showing a change in a color of Ettlia sp. YC001 according to culture time and culture conditions;

FIG. 9 is a diagram showing comparison of contents of chlorophyll and anthocyanin extracted from Ettlia sp. YC001 changed to green and red;

FIG. 10 shows TLC analysis results for various types of carotenoid and pigments extracted from Ettlia sp. YC001 changed to red;

FIG. 11 shows HPLC analysis results for various types of carotenoid extracted from Ettlia sp. YC001 changed to green and red;

FIG. 12 shows HPLC analysis results of contents of various types of carotenoid extracted from Ettlia sp. YC001 changed to green and red;

FIG. 13 shows peaks at retention times of 26.720 min and 35.613 min by analyzing Ettlia sp. YC001 changed to red through HPLC at 200 to 600 nm; and

FIG. 14 shows composition ratios of fatty acids in Ettlia sp. YC001 changed to red.

### MODES OF INVENTION

The inventors have completed the present invention as a result of a study on an industrially useful microalgae strain having high carbon dioxide fixability and a high lipid content.

To separate a high microalgae strain, the inventors collected environment samples from various environments, and then a new microalgae strain having increased biomass productivity, highly efficient carbon dioxide fixability and lipid productivity was separated.

As a result of morphological and molecular-biological identification, the microalgae strain was identified as Ettlia sp., and deposited in the biological resource center of the Korean Research Institute of Bioscience and Biotechnology (KRIBB) under Accession No. KCTC 12109BP.

Accordingly, the present invention provides an Ettlia sp. strain deposited under Accession No. KCTC 12109BP that has a high carbon dioxide fixation rate and a high biomass productivity, and a high lipid content.

Generally, the microalgae conducts photosynthesis using carbon dioxide as a carbon source, but when a high concentration of carbon dioxide is continuously provided, pH of a culture solution is decreased, and thus the microalgae cannot be properly grown. In addition, generally, when a concentration of carbon dioxide is increased, the microalgae having a resistance to the concentration of carbon dioxide are increased in growth rate and decreased in lipid content.

However, in one exemplary embodiment of the present invention, it was confirmed that Ettlia sp. YC001 (KCTC 12109BP) has no great changes in biomass and lipid contents according to the concentration of carbon dioxide, and a constantly maintained growth rate due to resistance ranging from pH 6 to pH 11, and compared to the lipid content of general microalgae is 16 to 23%, the lipid content of the Ettlia sp. strain is three times as high at 30 to 67% of a dry weight (refer to Example 2).

In addition, in another exemplary embodiment of the present invention, it was confirmed that the lipid content can be increased by controlling the culture conditions and/or culture time of Ettlia sp. YC001 (KCTC 12109BP), and a ratio of the C16 to C18 contents can be increased by controlling a composition ratio of a fatty acid (refer to Examples 2 and 3). The result shows that qualified biodiesel can be produced by controlling the culture conditions and/or culture time of the strain of the present invention.

In such an aspect, the present invention provides a composition for producing biodiesel including the strain or a homogenate thereof.

Meanwhile, here, there is no particular limit to the culture conditions of Ettlia sp. YC001 to produce biodiesel, but Ettlia sp. YC001 may be cultured for 3 to 60 days while carbon dioxide is provided at a concentration of 15 vol % or less, and preferably cultured for 5 to 20 days while carbon dioxide is provided at a concentration of 5 vol %.

In another exemplary embodiment of the present invention, a color of Ettlia sp. YC001 cells (KCTC 12109BP) changed from green to red when the culture conditions and/or culture time were controlled, and it was confirmed that the change of the cell color is caused by accumulating useful materials such as a pigment and an antioxidant material such as carotenoid in the cells (Example 4). According to the result, it was confirmed that a content of the carotenoid or pigment of the Ettlia sp. YC001 (KCTC 12109BP) of the present invention can be increased by controlling the culture conditions and/or culture time, and thus there is a high probability of utilization as bio resources such as foods, cosmetics, medicines, etc.

Accordingly, the present invention provides a composition for producing carotenoid including the strain or a homogenate thereof, as well as a composition for foods and a composition for cosmetics, which include the strain or a homogenate thereof.

Here, there is no particular limit to the culture conditions of the Ettlia sp. YC001 strain included in the compositions, but the Ettlia sp. YC001 strain may be cultured for 3 to 60 days, preferably 20 days or more, and more preferably 30 days or more.

The composition for foods of the present invention includes Ettlia sp. YC001 or a homogenate thereof as an essential component, and may be contained at 0.01 to 95 wt %, and preferably 1 to 80 wt %, with respect to a total weight of the composition, but is not limited thereto.

There is no particular limit to components included in the composition for foods, other than the strain or a homogenate thereof, and generally, the composition for foods may contain various types of aromas, natural hydrocarbons, nutrients, vitamins, minerals (electrolytes), flavoring agents such as synthetic and natural flavoring agents, coloring agents, enhancers (cheese, chocolate, etc.), pectic acid and salts thereof, alginic acid and salts thereof, organic acids, protective colloid thickening agents, pH adjusters, stabilizers, preservatives, glycerin, alcohols, or carbonating agents used in carbonated soft drinks. In addition, the composition of the present invention may contain fruit pulp to prepare natural fruit juice, fruit juice beverages, and vegetable beverages. Such components may be used independently or in combination, and there is no limit to their contents, but they may be included at, for example, 0.001 to approximately 20 parts by weight with respect to 100 parts by weight of the composition of the present invention.

The composition for cosmetics of the present invention also includes Ettlia sp. YC001 or a homogenate thereof as an essential component. The strain or a homogenate thereof may be contained at 0.01 to 95 wt %, and preferably 1 to 80 wt %, with respect to a total weight of the composition, but the present invention is not limited thereto. As another component for the composition for cosmetics, at least one of the components generally used in compositions for cosmetics may be used.

In addition, the composition for cosmetics may be prepared in any type of a liquid, a cream, a paste, and a solid according to its use, by a general method, and for example, may be prepared as an astringent, an emollient, an emulsion, a massage cream, an essence, a pack, a lotion, a cream, etc.

When the composition for cosmetics of the present invention is an emulsion type, in addition to the strain of the present invention or a homogenate thereof, distilled water, a monohydric or polyhydric alcohol, a fatty acid, an oil and a surfactant may be included, and other fragrance ingredients, coloring agents, or preservatives may be used. When the composition for cosmetics of the present invention is solubilized, in addition to the strain of the present invention or a homogenate thereof, distilled water, a surfactant, or a monohydric or polyhydric alcohol may be included as additional components. In addition, when the composition for cosmetics of the present invention is an emulsion type, a fragrance ingredient, a coloring agent or a preservative may be used as an additional component, and when the composition for cosmetics of the present invention is prepared as a cream, a plant extract is contained in a general oil-in-water (O/W) type cream base, a fragrance, a chelating agent, a pigment, an antioxidant, and a preservative may be added, and synthetic or natural materials such as a protein, a mineral, a vitamin, etc. may also be used to improve physical properties.

Hereinafter, exemplary Examples will be provided to help in understanding the present invention. However, the following examples are merely provided such that the present invention can be more easily understood, not to limit the scope of the present invention.

### EXAMPLES

### Example 1

### Separation of Microalgae from Environment Samples

To separate microalgae having excellent biomass productivity and a high lipid content from environment samples, environment samples were collected. As the environment samples, soil obtained from a peripheral region of a microalgae mass culture system located in Yuseong-gu, Daej eon Metropolitan City, soil obtained from a peripheral region of Gapcheon in Daejeon Metropolitan City, and a sample obtained from Miwami pond in Jeju Island were used. The environment samples were suspended in distilled water, and centrifuged at 4,000 rpm for 10 minutes, and then a supernatant was removed. In addition, the resulting pellets were suspended with distilled water, diluted at a concentration of 1/10 to 1/10⁴, plated on BG11 solid media, and cultured at 25° C. and a luminous intensity of 120 μmol photons/m²/s until green colonies were shown. The components of the BG11 used herein are shown in Table 1.

TABLE 1

| Medium Components | Content (mg/L) |
|---|---|
| NaNO₃ | 1500 |
| K₂HPO₄ | 39 |
| MgSO₄•7H₂O | 75 |
| Na₂CO₃ | 21 |
| CaCl₂ | 27 |
| Ferric citrate | 6 |
| Citric acid | 6 |
| Na₂EDTA | 1 |
| Microelement | 1 (ml/L) |
| microelement (mg/500 ml) | 286018102223917949.4 |
| H₃BO₃MnCl₂•4H₂OZnSO₄•7H₂ONa₂MoO₄•2H₂OCuSO₄•5 | |
| H₂OCo(NO₃)₂•5H₂O | |

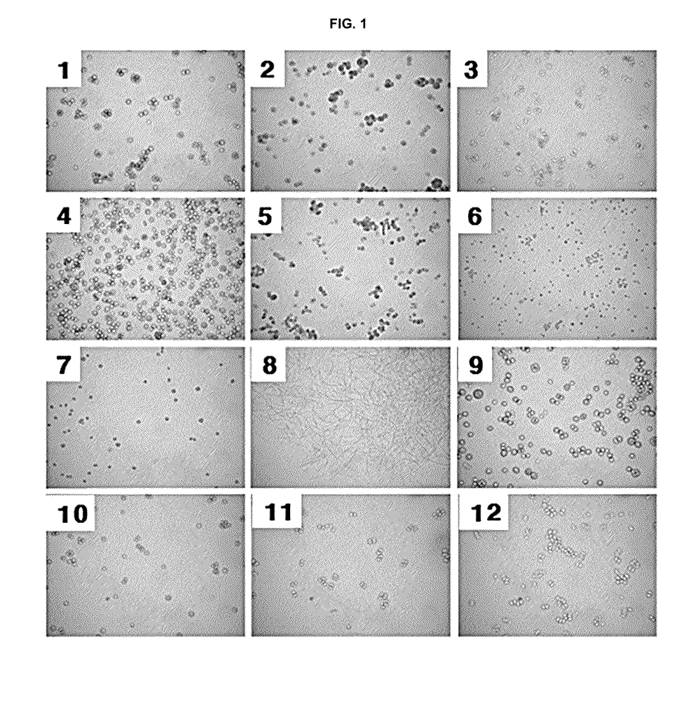

One hundred single green colonies generated in the BG11 solid medium were inoculated into a BG11 liquid medium, and cultured for 16 days under the same conditions as described above. The cultured colonies were observed with an optical microscope. The result is shown in FIG. 1.

As shown in FIG. 1, 12 types of single microalgae strains having different morphologies were separated. 12 types of the separated single microalgae strains were cultured in 24-well micro plate containing BG11 liquid media, and four strains thereof having high chlorophyll concentrations were selected to be cultured under a condition in which 10 vol % of carbon dioxide was provided at 0.3 v/v/m.

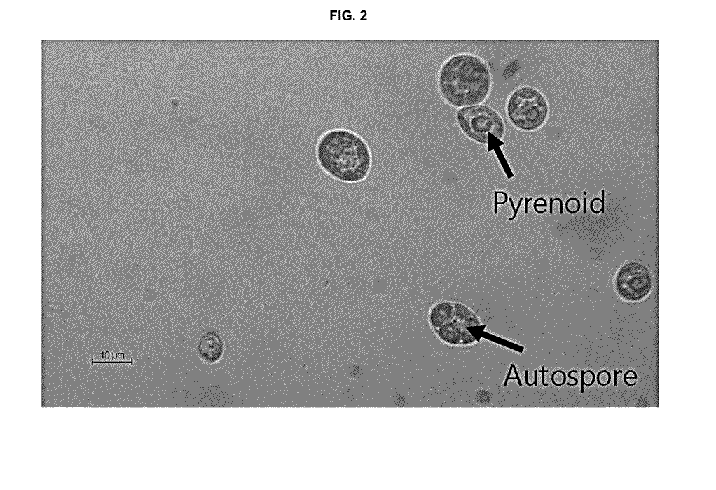

Among the four types of the microalgae strains cultured by providing 10 vol % carbon dioxide, the microalgae strain having the highest biomass productivity was separated as a single microalga using fluorescence activated cell sorter (FACS), and morphological characteristics of the strain were identified using an optical microscope. The result is shown in FIG. 2.

As shown in FIG. 2, it was confirmed that the microalga had a spherical shape having a diameter of 9 to 11 μm, one pyrenoid in a cell, produced an autospore and an endospore, and thus was divided by sporulation.

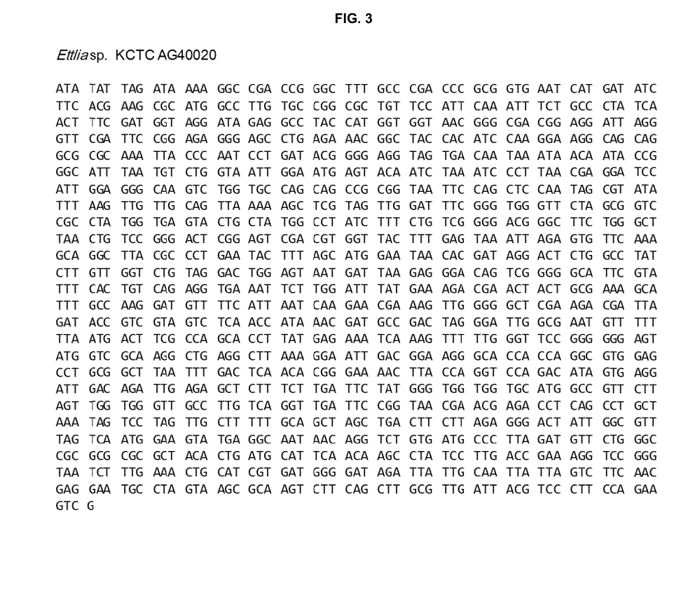

In addition, to identify the microalga by a molecular-biological technique, 18S rDNA was amplified using 165F (5′-CGA CTT CTG GAA GGG ACG TA-3′, SEQ. ID. NO: 1) forward primer and 1780R (5′-CTA GGT GGG AGG GTT TAA TG-3′, SEQ. ID. NO: 2) reverse primer through polymerase chain reaction (PCR). The PCR was performed by repeating a procedure including denaturation (94° C., 1 min), binding (58° C., 1 min), and polymerization (72° C., 1 min) 30 times. A product obtained by PCR was analyzed by an ABI 3730XL sequencer, thereby obtaining an 18S rDNA sequence (SEQ. ID. NO: 3). The result is shown in FIG. 3.

As the result of analyzing the 18S rDNA sequence with NCBI database, it was confirmed that it had a homology of 98% with the base sequence of a microalgae strain, Ettlia sp., and it was confirmed that the strain was included in the same group with Ettlia sp. through phylogenetical analysis. According to the results, the separated strain was identified as Ettlia sp. through morphological/molecular biological analyses, and deposited in the biological resource center of the KRIBB under Accession No. KCTC 12109BP.

### Example 2

### Confirmation of Growth and Lipid Content of Ettlia sp. YC001 (KCTC 12109BP) Under Various Concentrations of Carbon Dioxide

Generally, the microalgae conducted photosynthesis using carbon dioxide as a carbon source, and when a high concentration of carbon dioxide was consistently provided, a pH of a culture solution decreased, and thus the microalgae could not be properly grown. In addition, the strain that can be grown in a high concentration of carbon dioxide is generally decreased in lipid content, and thus decreased in lipid productivity.

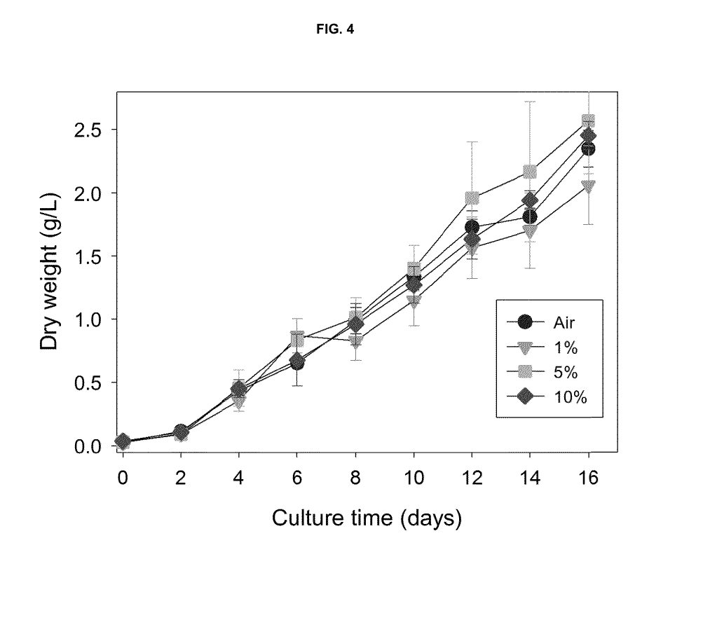

Accordingly, to confirm growth of Ettlia sp. YC001 (KCTC 12109BP) separated in Example 1 with various concentrations of carbon dioxide, the air and 1, 5 or 10 vol % of carbon dioxide were provided at 0.1 v/v/m, and the strain was cultured at 26±1° C. and 120 μmol photons/m²/s for 16 days. To confirm a concentration of the cells during culture, the Ettlia sp. YC001(KCTC 12109BP) culture solution was filtered using a filter previously dried at 105° C., cells remaining on the filter were dried at 105° C. for 12 hours, and then a dry weight was measured. The result is shown in FIG. 4.

As shown in FIG. 4, Ettlia sp. YC001 (KCTC 12109BP) was grown at 2 g/L in every condition. Particularly, the maximum cell concentration and biomass productivity had highest values at 2.57 g/L and 0.28 g/L/d under a condition of 5 vol % carbon dioxide, respectively. In addition, pH of the culture solution in the early stage of culture was 6.3, and increased to 10 to 11 as the microalgae were grown. However, it was confirmed that, regardless of the change and increase in pH, a growth rate of Ettlia sp. YC001 (KCTC 12109BP) was constantly maintained. The result showed that Ettlia sp. YC001 (KCTC 12109BP) had resistance to pH change in a wide range and was cultured for a long time.

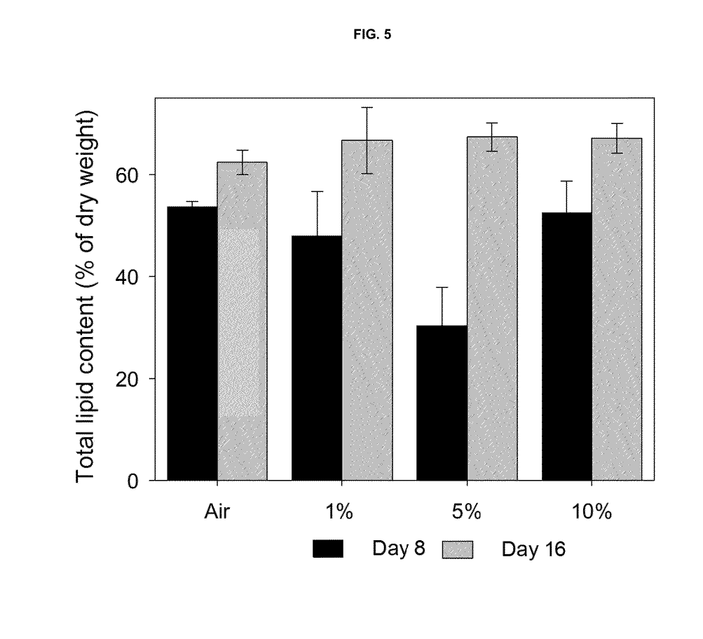

In addition, to confirm a lipid content of Ettlia sp. YC001 (KCTC 12109BP) with the various concentrations of carbon dioxide, the lipid content of the strain cultured under the same conditions was analyzed according to a culture time through chloroform-methanol analysis. The result is shown in FIG. 5.

As shown in FIG. 5, on day 8 of the culture, when the air was provided, the lipid content was the highest at 54 wt % of the dry weight, and when 5% of carbon dioxide was provided, the lipid content was the lowest at 30 wt % of the dry weight. However, it was confirmed that, on day 16 of the culture, regardless of the concentration of carbon dioxide, the lipid content was increased at 60 wt % or more.

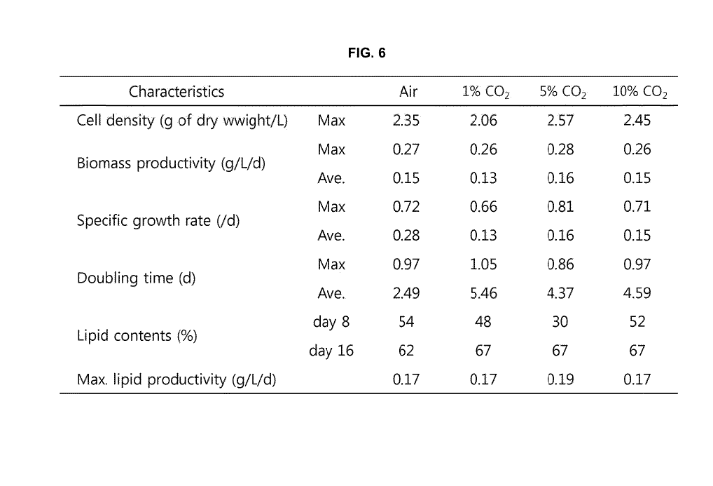

In addition, the lipid productivity was calculated using the following equation. The result is shown in FIG. 6.

Lipid productivity (g/L/d)=Biomass productivity (g/L/d)×Lipid content (wt %)/100

As shown in FIG. 6, the maximum lipid productivity was 0.19 g/L/d, and 5 vol % of carbon dioxide was provided. In addition, when 5 vol % of carbon dioxide was provided, as well as the lipid productivity, cell density, biomass productivity and lipid content were also the highest.

### Example 3

### Confirmation of Composition Ratio of Fatty Acid of Ettlia sp. YC001 (KCTC 12109BP)

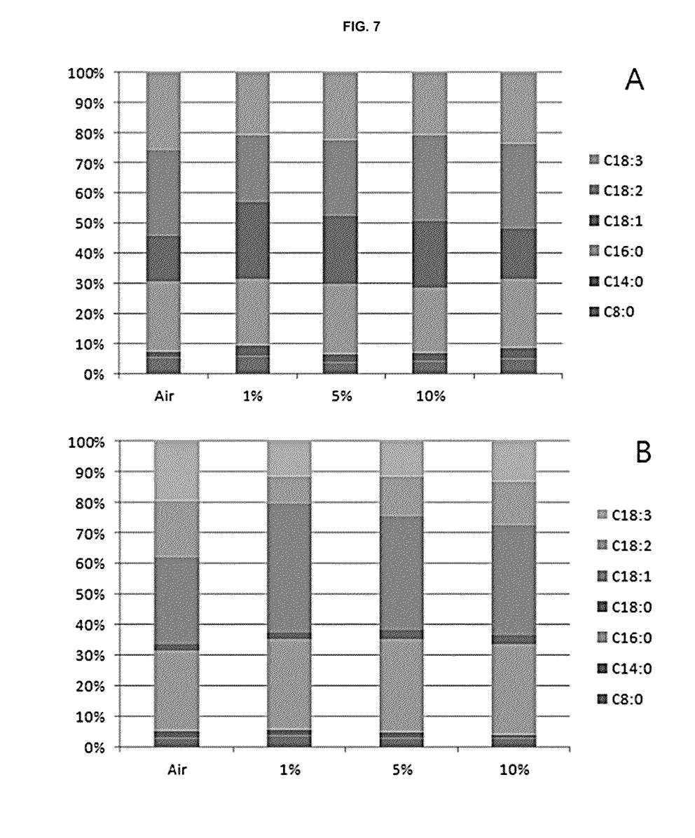

The composition ratio of a fatty acid is the most important factor in the quality of biodiesel, and having more fatty acids having 16 to 18 carbon atoms is advantageous for producing high quality biodiesel. Particularly, according to a conventional study, an 18:1 fatty acid is advantageous for producing high quality biodiesel. Accordingly, to confirm whether the Ettlia sp. YC001 (KCTC 12109BP) of the present invention can also be used to produce high quality biodiesel, a composition ratio of the fatty acid according to a culture time was confirmed. The composition ratio of the fatty acid was analyzed using gas chromatography. The result is shown in FIG. 7.

As shown in FIG. 7A, it was confirmed that, on day 8 of the culture, all of 16:0, 18:1, 18:2, and 18:3 had similar ratios. However, as shown in FIG. 7B, it was confirmed that, on day 16 of the culture, the 16:0 insignificantly increased, but the 18:1 fatty acid was 40% or more when carbon dioxide was provided.

According to the result, it was seen that the Ettlia sp. YC001 (KCTC 12109BP) of the present invention can produce high quality biodiesel when the culture time and/or culture conditions are controlled.

### Example 4

### Observation of Physiological Change of Cells According to Carotenoid Accumulation

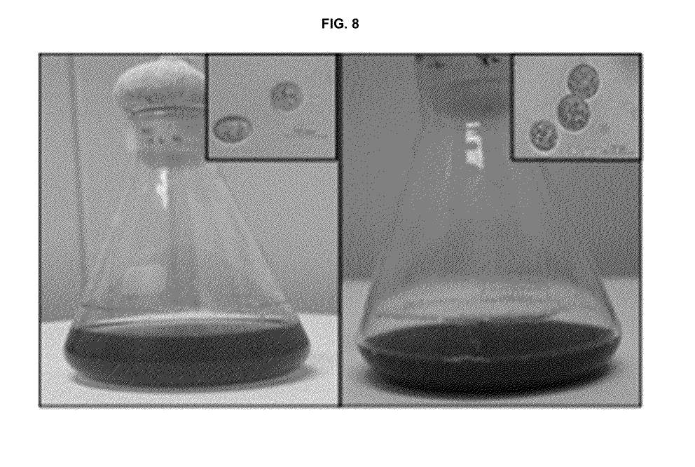

Ettlia sp. YC001 (KCTC 12109BP) was inoculated into a triangle flask containing 150 ml of BG11 media and cultured for 30 days at 25° C. and 120 μmol photons/m²/s. Afterward, the shape and color of cells were checked, and morphological characteristics of green and red cells were observed with an optical microscope. The results are shown in FIG. 8.

As shown in FIG. 8, the change in the color of the cells from green to red could be observed along with internal changes of the cells. It was observed that, due to the depletion of nutrient salts in a culture solution according to the culture time, the cells of the present invention changed into cyst cells. As shown in FIG. 8, it was also observed that the green cells had a circular or oval shape, but the red cells had only a circular shape. Autospores and pyrenoids observed in the green cells were not observed in the red cells, but endospores were observed in the cells. As a result, it was confirmed that the cells changed not only in color, but also morphologically.

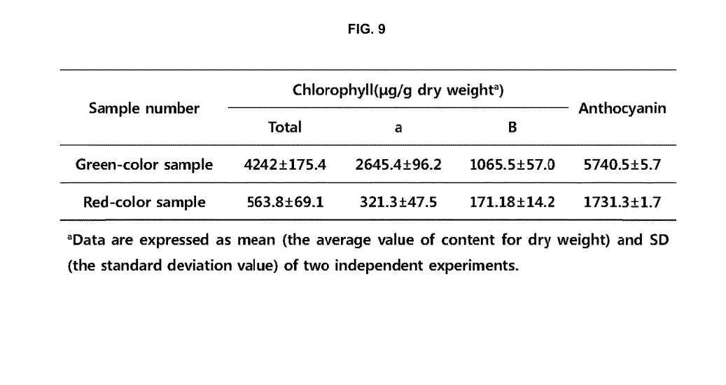

In addition, when the color of the cells had changed, the change of a pigment in the cells was analyzed, which is shown in FIG. 9.

As shown in FIG. 9, a total chlorophyll concentration in the green cells was 4242±175.4 μg/g (dry weight), but a total chlorophyll concentration in the red cells was 563.8±69.1 μg/g (dry weight), which was decreased approximately 86% or more, compared to when there were no changes in the color of the cells. It was also confirmed that contents of chlorophyll a and b were decreased approximately 87% and 84% or more, respectively.

A concentration of anthocyanin was 5740 μg/g (dry weight) in a green sample, but 1731 μg/g (dry weight) in a red sample. It was reported that large amounts of anthocyanin were accumulated when the color of leaves of the plant generally changed from green to red. However, the anthocyanin content in the cells of the present invention was considerably decreased, compared to that in the green cells, and thus it was confirmed that the change in the color of the cells was not caused by anthocyanin.

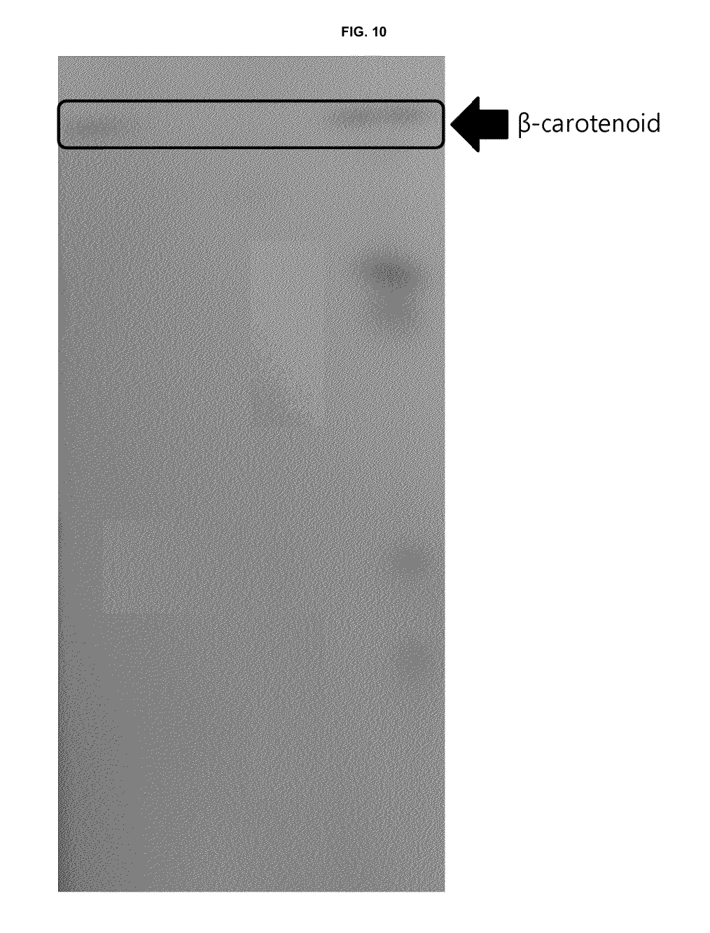

To confirm whether the color change was caused by carotenoid, carotenoid and a pigment were extracted from the red sample using acetone, and thin-layer chromatography (TLC) was performed using a representative carotenoid, that is, β-carotene, as a standard material. The result is shown in FIG. 10.

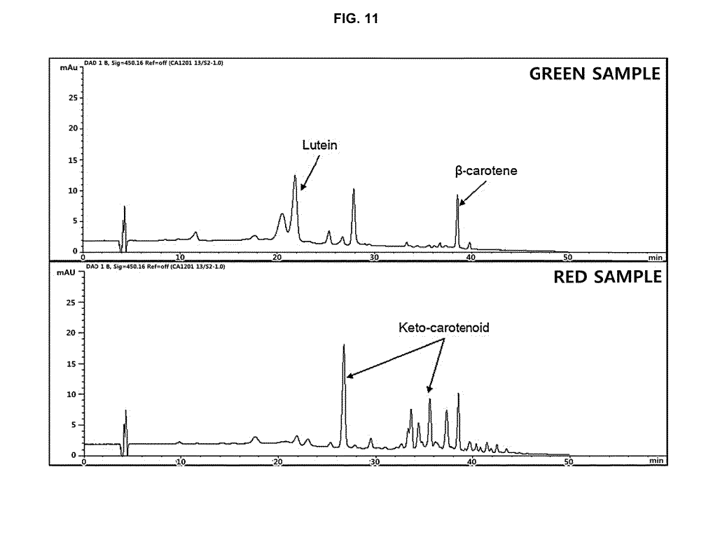

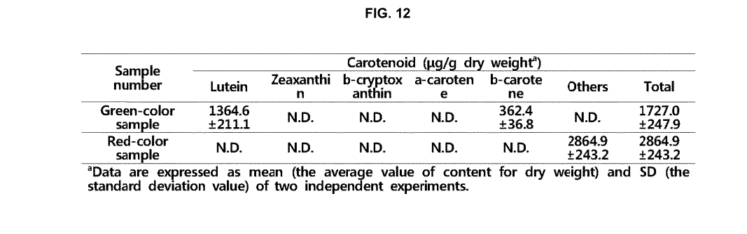

As shown in FIG. 10, β-carotene was not detected but various types of pigments and carotenoid were detected from the red sample. For exact analysis of the carotenoid, carotenoids and pigments were extracted from acetone in green and red samples, and qualitative and quantitative analyses were performed through high performance liquid chromatography (HPLC). The results are shown in FIGS. 11 and 12.

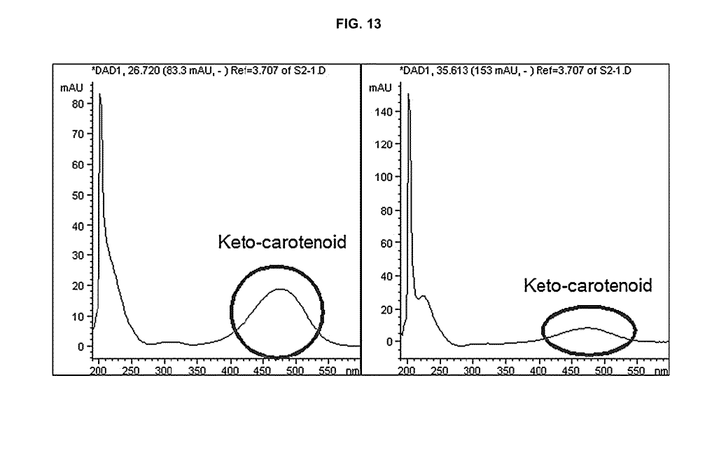

As shown in FIG. 11, according to the HPLC results, lutein and β-carotene were detected from the green sample, but not from the red sample. In addition, as shown in FIG. 12, lutein and β-carotene in the green sample were contained at 1364.6±211.1 μg/g (dry weight) and 362.4±36.8 μg/g (dry weight), respectively, and materials corresponding to standard materials including lutein and β-carotene could not be detected in the red sample. However, a large amount of carotenoid was detected in the red sample, as well as the standard materials, a total carotenoid content was 1727±247.9 μg/g (dry weight) in the green sample, and 2864.9±243.2 μg/g (dry weight) in the red sample, which was 1.6 times higher than that in the green sample. As the result of analyzing peaks of the red sample shown in FIG. 11 having retention time of 26.720 min and 35.613 min at a wavelength of 200 to 600 nm through HPLC, as shown in FIG. 13, one peak was detected at 450 to 500 nm, which is assumed to be an antioxidant, for example, a keto-carotenoid-based material.

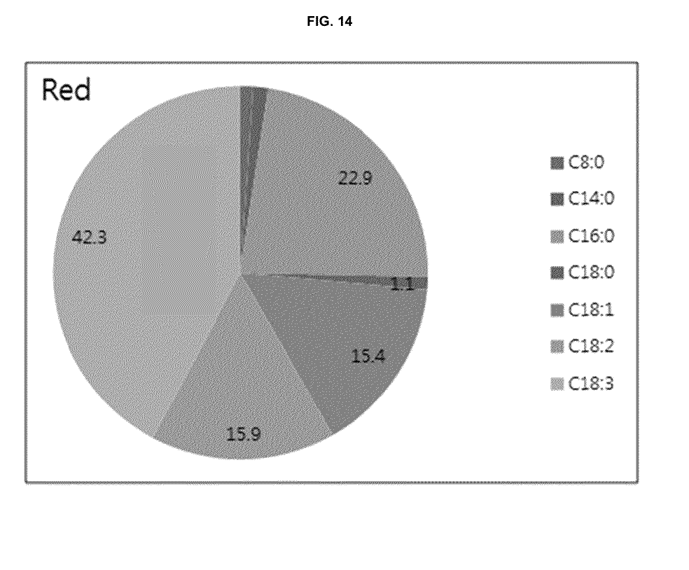

The change in a composition ratio of a fatty acid was confirmed by the same method as described in Example 3. The result is shown in FIG. 14.

As shown in FIG. 14, it was confirmed that a composition ratio of C18:3 was 20.0% in the green cells, but increased to 42.3% in the red cells. Accordingly, it was confirmed that the change in color of the cell was not only a morphological change but also a physiological change.

As a result, it was confirmed that high quality biodiesel, and additional useful materials such as an antioxidant can be produced using the cells by controlling the culture conditions and/or culture time.

Consequently, it was confirmed that Ettlia sp. YC001 (KCTC 12109BP) of the present invention is a cell having a high resistance to environmental stress, and higher biomass (0.28 g/L/day) and lipid (67%) contents than general microalgae, and can be used as a strain for producing high quality biodiesel in which the C16 and C18 contents are 60% of the total content of fatty acids. In addition, apparent discrimination of morphological characteristics and differentiation of the cells according to the concentration of carbon dioxide and the culture time means that Ettlia sp. YC001 can be used in various physiological and genetic engineering studies for microalgae.

Ettlia sp. YC001 (KCTC 12109BP) of the present invention has a very high photosynthesis efficiency, and high carbon dioxide reducing efficiency and biomass productivity, and thus can be used as a strain for producing high quality biodiesel by controlling culture conditions and/or culture time, and producing additional useful materials including an antioxidant such as carotenoid, and further can be used as various biomaterials such as foods, cosmetics, etc. Moreover, since morphological characteristics and a differentiation process of the cells are apparently different according to a concentration of carbon dioxide and culture time, Ettlia sp. YC001 can be used in various physiological and genetic engineering studies for microalgae.

While the invention has been shown and described with reference to certain exemplary embodiments thereof, it will be understood by those skilled in the art that various modifications in form and details may be made therein without departing from the spirit and scope of the invention as defined by the appended claims.

1. An Ettlia sp. strain, which was deposited under Accession No. KCTC 12109BP.

2. The strain according to claim 1, which has an 18S rDNA sequence of SEQ. ID. NO: 3.

3. The strain according to claim 1, which has a lipid content of 30 to 67% of a dry weight.

4. The strain according to claim 1, which has carotenoid productivity.

5. The strain according to claim 1, which has a resistance in a range from pH 6 to pH 11.

6. The strain according to claim 1, which is cultured under a condition of 15 vol % or less of carbon dioxide.

7. The strain according to claim 1, which is cultured for 3 to 60 days.

8. A composition for producing biodiesel, comprising:
the strain of claim 1 or a homogenate thereof.

9. A composition for producing carotenoid, comprising:
the strain of claim 1 or a homogenate thereof.

10. A composition for foods, comprising:
the strain of claim 1 or a homogenate thereof.

11. A composition for cosmetics, comprising:
the strain of claim 1 or a homogenate thereof.


SEQUENCE LISTING (3 sequences)

SEQ ID NO: 1 - DNA, 20 bp, Artificial Sequence
  165F forward primer
cgacttctggaagggacgta

SEQ ID NO: 2 - DNA, 20 bp, Artificial Sequence
  1780R reverse primer
ctaggtgggagggtttaatg

SEQ ID NO: 3 - DNA, 1429 bp, Ettlia sp.
atatattagataaaaggccgaccgggctttgcccgacccgcggtgaatcatgatatcttcacgaagcgcatggccttgtgccggcgctgttccattcaaatttctgccctatcaactttcgatggtaggatagaggcctaccatggtggtaacgggcgacggaggattagggttcgattccggagagggagcctgagaaacggctaccacatccaaggaaggcagcaggcgcgcaaattacccaatcctgatacggggaggtagtgacaataaataacaataccgggcatttaatgtctggtaattggaatgagtacaatctaaatcccttaacgaggatccattggagggcaagtctggtgccagcagccgcggtaattccagctccaatagcgtatatttaagttgttgcagttaaaaagctcgtagttggatttcgggtgggttctagcggtccgcctatggtgagtactgctatggcctatctttctgtcggggacgggcttctgggcttaactgtccgggactcggagtcgacgtggttactttgagtaaattagagtgttcaaagcaggcttacgccctgaatactttagcatggaataacacgataggactctggcctatcttgttggtctgtaggactggagtaatgattaagagggacagtcgggggcattcgtatttcactgtcagaggtgaaattcttggatttatgaaagacgaactactgcgaaagcatttgccaaggatgttttcattaatcaagaacgaaagttgggggctcgaagacgattagataccgtcgtagtctcaaccataaacgatgccgactagggattggcgaatgtttttttaatgacttcgccagcaccttatgagaaatcaaagtttttgggttccggggggagtatggtcgcaaggctgaggcttaaaggaattgacggaagggcaccaccaggcgtggagcctgcggcttaatttgactcaacacgggaaaacttaccaggtccagacatagtgaggattgacagattgagagctctttcttgattctatgggtggtggtgcatggccgttcttagttggtgggttgccttgtcaggttgattccggtaacgaacgagacctcagcctgctaaatagtcctagttgctttttgcagctagctgacttcttagagggactattggcgtttagtcaatggaagtatgaggcaataacaggtctgtgatgcccttagatgttctgggccgcgcgcgcgctacactgatgcattcaacaagcctatccttgaccgaaaggtccgggtaatctttgaaactgcatcgtgatggggatagattattgcaattattagtcttcaacgaggaatgcctagtaagcgcaagtcttcagcttgcgttgattacgtcccttccagaagtcg